(19) United States
(12) Patent Application Publication
(10) Pub. No.: US 2003/0013690 A1
(43) Pub. Date: Jan. 16, 2003

(54) Increasing phosphorus uptake from the gut of dairy cows by supplementing 1alpha-hydroxylated vitamin D compounds

(76) Inventors: Hector F. DeLuca, Deerfield, WI (US); Neal A. Jorgenson, Middleton, WI (US); Larry D. Satter, Madison, WI (US)

Correspondence Address: Thomas M. Wozny, ANDRUS, SCEALES, STARKE & SAWALL, LLP, Suite 1100, 100 East Wisconsin Avenue, Milwaukee, WI, 53202-4178, US

(21) Appl. No.: 09/815,573

(22) Filed: Mar. 22, 2001

(51) Int. Cl. A61K 31/59

(52) U.S. Cl. 514/167

(57) ABSTRACT

A feed for dairy cows containing 1α-hydroxylated vitamin D compounds. The vitamin D compounds cause improved utilization of dietary phosphorus and can eliminate the need for supplemental quantities of inorganic phosphorus in the cows' diet. In particular, milk production in dairy cows may be maintained in relatively high yield even though fed a low P diet.

### BACKGROUND OF THE INVENTION

An animal requires phosphorus (P) for formation of bones and teeth, for phopholipid (cell membrane structure), for nucleic acid (RNA, DNA) synthesis, for synthesis of ATP and other high-energy P compounds, and for proper acid/base balance. In particular, lactating dairy cows require sufficient P in their diets in order to maintain adequate milk yield.

Up to 80% of the P present in plant foods and feeds exists as a complex of phytic acid (myoinositol hexaphosphate), hereafter referred to as phytate. Phytate may structurally be illustrated by the following formula:

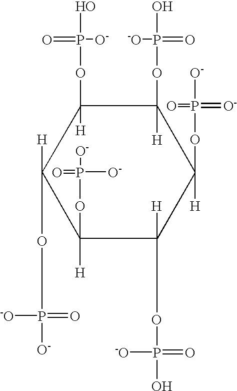

The P in phytate is largely unavailable to simple-stomached animals, including humans, and therefore, it passes through the GI tract and is excreted in the feces. In swine and poultry nutrition, this is accounted for in diet formulation whereby 1.5-2.0% of an inorganic phosphate source is supplemented to meet the animal's minimal P requirement.

Supplemental inorganic P is provided to animal diets in one of three feed-grade forms: dicalcium phosphate (18.5% P), monocalcium phosphate (21.5% P) or deflorinated phosphate (18.0% P). The combined total market for these products is estimated to be 675 million dollars per year in the U.S., Canada, Mexico, Western Europe and Japan. If one were to include South America, Eastern Europe, Asia, Africa, China, India, and Southeast Asia, (where market data are difficult to obtain), the total market for feed-grade phosphates could easily be expected to exceed 1 billion dollars annually. Thus, supplemental inorganic P is a relatively expensive ingredient in an animal's diet. It is often stated that P is the third most expensive dietary ingredient, after energy and protein. As a result, its reduction and/or elimination would be desirable from a cost standpoint. Preferably, this reduction in the need for supplemental quantities or inorganic P should be accomplished by increasing the utilization of organic P inherently present in animal feed. In dairy cows, however, such a reduction of inorganic P cannot be made at the expense of milk yields.

Reducing dietary inorganic P would also reduce the P content of manure. Animal manure, as well as human waste, is generally spread on agricultural land, where a portion of the P gets into surface runoff and then into ponds, streams, rivers, lakes, and oceans. Too much P in water stimulates growth of algae, and algae take up considerable oxygen. This robs marine life of the oxygen they need to grow, reproduce, and thrive. In many parts of Europe and Asia, P pollution has become such a problem and concern that penalties in the form of stiff financial fines are imposed on livestock producers who spread too much P-laden manure on the soils. Many U.S. soils are being described as “P-saturated”, thus resulting in a greater concentration of P in soil leachates and surface runoff. High-P water leachate in areas such as the Chesapeake Bay has been blamed for excessive algae growth and increased fish kills in bay waters (Ward, 1993). In Europe, the feed industry group FEFANA issued a position paper in 1981 entitled “Improvement of the Environment.” This group proposed that P in manure from livestock production should be reduced by 30% (Ward, 1993). The limits of P that can be applied to soils in Europe have been discussed by Schwarz (1994). Accordingly, it is desirable to provide a method and/or feed composition that would reduce the P content of animal waste products.

Under normal dietary circumstances, cholecalciferol (vitamin D₃) that is added to a diet gets absorbed from the gastrointestinal (GI) tract and is transported via blood to the liver where the liver enzyme, 25-hydroxylase, acts on the compound to cause formation of 25-OH D₃. This compound is the normal blood metabolite of cholecalciferol. A small portion of 25-OH D₃ undergoes a further hydroxylation step in the kidney at the 1-α position, causing synthesis of the calciotrophic hormone 1,25-(OH)₂D₃.

Edward's U.S. Pat. No. 5,366,736 showed that in monogastric animals such as swine and fowl, the compound 1,25-(OH)₂D₃ is effective in improving P utilization from phytate-bound P, and Biehl et al. (1995) confirmed Edward's results. Moreover, both studies showed that 1,25-(OH)₂D₃ works additively with microbial phytase in releasing P from dietary phytate complexes. Neither references, however, discussed the effect on inorganic P or the possible effect on lactating dairy cows. It seems likely that 1,25-(OH)₂D₃ exerts its effects in two ways: (a) the 1,25-(OH)₂D₃ compound likely increases the activity of intestinal phytases or phosphatases that hydrolyze phytate (Pileggi et al., 1995; Maddaiah et al., 1964), and (b) the 1,25-(OH)₂D₃ compound is known to stimulate phosphate transport (Tanka and DeLuca, 1974), facilitating transport of P from the GI tract to plasma and hence bone.

Phytate complexes in plant foods and feeds (e.g., cereal grains and beans) also bind cations such as zinc, iron, and manganese (Erdman, 1979). This is illustrated schematically as follows:

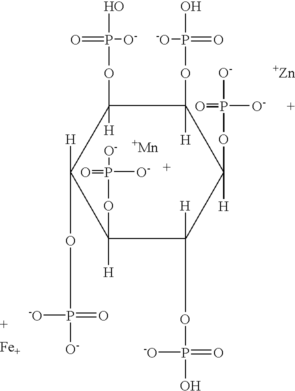

In addition, these three trace elements are always added in supplemental form to diets for ruminant animals as feed-grade ZnO or ZnSO₄.H₂O, FeSO₄.H₂O, MnO or MnSO₄.H₂O. Again, it would be desirable from a cost standpoint as well as an environmental standpoint to provide a method and/or feed composition that increases utilization of these elements so as to also reduce the need for supplemental quantities of such minerals in an animal's diet.

In ruminant animals such as dairy cows the large population of bacteria and protozoa in the first compartment of the four-compartment stomach produce phytase, and it is generally accepted that ruminant animals utilize a large proportion of phytate P in the diet. It is likely that in the present invention that 1-α-OH vitamin D is increasing P uptake from the ruminant primarily by stimulating phosphate transport across gut membranes. The impact of 1-α-OH vitamin D on increased P uptake from the gut through action on intestinal phytases or phosphotases is likely to be of secondary importance.

### SUMMARY OF THE INVENTION

It is an object of the present invention to provide a feed supplement for a dairy cow that eliminates or at least substantially reduces the need for supplemental inorganic P in the cow's diet.

It is another object of the present invention to provide a bioactive feed additive that increases utilization of P from phytate.

It is yet another object of the present invention to provide a method of maintaining milk production in a dairy cow while at the same time minimizing the need for supplemental inorganic P and increasing utilization of phytate P in the cow's diet.

It is a further object of the present invention to provide a bioactive feed additive that also increases utilization of other minerals such as Zn, Mn, and Fe from phytate.

In accordance with the above objects of the invention, a feed supplement for a dairy cow includes an effective amount of 1α-hydroxylated vitamin D compound. The preferred vitamin D compounds are 1α-hydroxyvitamin D₃ or 1α,25-dihydroxyvitamin D₃. The 1α-hydroxylated vitamin D compounds are incorporated into the feed additive so as to provide about 0.1 μg/kg to about 100 μg/kg of feed in the cow's diet. By incorporating a 1α-hydroxylated vitamin D compound in the diet of a dairy cow, the feed can be formulated with only about 0.3% by weight or less of inorganic P supplements, and preferably with no inorganic P supplementation.

Accordingly, the present invention provides a method of compounding feed for a dairy cow, comprising the steps of providing a feed supplement for a dairy cow that contains about 0.3% by weight or less of an inorganic phosphorus supplement; incorporating with said feed supplement an effective amount of a 1α-hydroxylated vitamin D compound to form a feed mixture; and forming said feed mixture into a discrete shape.

The present invention also provides an animal feed composition for a diary cow comprising a feed supplement that contains about 0.3% by weight or less of an inorganic phosphorus supplement; and an effective amount of an 1α-hydroxylated vitamin D compound for increasing phosphorus uptake in a cow's gut.

Further, the present invention provides a method of minimizing dietary requirements for phosphorus in a dairy cow, and more particularly, a method of maintaining milk production at normal yields in dairy cattle fed a low P diet, comprising the steps of feeding a feed that contains about 0.3% by weight or less of an inorganic phosphorus supplement to a diary cow; and feeding with said feed an effective amount of a 1α-hydroxylated vitamin D compound for increasing phosphorus uptake in a cow's gut.

By replacing some or all of the trace minerals (e.g. Zn, Mn and Fe) as well as inorganic P normally supplied in the diet as a supplement to dairy cattle, the remaining diet would contain more usable energy. Thus, grain-oilseed meals diets generally contain about 3,200 kcal metabolizable energy per kilogram in diet, and mineral salts supply no metabolizable energy. Removal of the unneeded minerals and substitution with grain would therefore increase the usable energy in the diet.

In summary, the potential benefits of the present invention include: (a) substantial reduction and/or elimination of the need for inorganic P supplements in diary cattle diets; (b) the maintenance of normal milk production in dairy cattle even though fed a low P diet; (c) reduction in P pollution of the environment; and (d) possible reduction in the need for supplemental Zn, Mn, and Fe in dairy cattle diets.

### DETAILED DESCRIPTION OF THE PREFERRED EMBODIMENT

As used in the description and in the claims, the term hydroxy-protecting group signifies any group commonly used for the temporary protection of hydroxy functions, such as for example, alkoxycarbonyl, acyl, alkylsilyl, and alkoxyalkyl group, and a protected hydroxy group is a hydroxy function derivatized by such a protecting group. Alkoxycarbonyl protecting groups are groupings such as methoxycarbonyl, ethoxycarbonyl, propoxycarbonyl, isopropoxycarbonyl, butoxycarbonyl, isobutoxycarbonyl, tert-butoxycarbonyl, benzloxycarbonyl or allyloxycarbonyl. The term “acyl” signifies an alkanoyl group of 1 to 6 carbons, in all of its isometric forms, or a carboxyalkanoyl group of 1 to 6 carbons, such as an oxalyl, amlonyl, succinyl, glutaryl group, or an aromatic acyl group such as benzoyl, or a halo, nitro or alkyl substituted benzoyl group. The word “alkyl” as used in the description or the claims, denotes a straight-chain or branched alkyl radical of 1 to 10 carbons, in all its isomeric forms. Alkoxyalkyl protecting groups are groupings such as methoxymethyl, ethoxyethyl, methoxyethoxymethl, or tetrahydrofuranyl and tetrahydropyranyl. Preferred alkylsilyl protecting groups are trimethylsilyl, triethylsilyl, t-butyldimethylsilyl, and analogous alkylated silyl radicals.

The vitamin D compounds useful in the present treatment are 1α-hydroxyated vitamin D compounds, preferably 1α-hydroxycholecalciferol. The vitamin D compounds of this type are characterized by the following general structure:

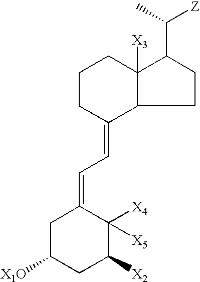

where X₁ may be hydrogen or a hydroxy-protecting group, X₂ may be hydroxy, or protected hydroxy, X₃ may be hydrogen or methyl, X₄ and X₅ each represent hydrogen or taken together X₄ and X₅ represent a methylene group, and where Z is selected from Y, —OY, —CH₂OY, —C≡CY and —CH═CHY, where the double bond may have the cis or trans stereochemical configuration and where Y is selected from hydrogen, methyl, —CR₅O and a radical of the structure:

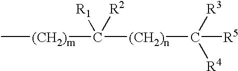

where m and n, independently, represent integers from 0 to 5, where R¹ is selected from hydrogen, hydroxy, protected-hydroxy, fluoro, trifluoromethyl, and C₁₋₅-alkyl, which may be straight chain or branched and, optionally, bear a hydroxy or protected-hydroxy substituent, and where each of R², R³ and R⁴, independently, is selected from hydrogen, fluoro, trifluoromethyl and C₁₋₅ alkyl, which may be straight-chain or branched, and optionally bear a hydroxy or protected-hydroxy substituent, and where R¹ and R², taken together, represent an oxo group, or an alkylidene group, ═CR₂R₃, or the group —CH₂)ₚ—, where p is an integer from 2 to 5, and where R³ and R⁴, taken together, represent an oxo group, or the group —(CH₂)_{q}—, where q is an integer from 2 to 5, and where R⁵ represents hydrogen, hydroxy, protected-hydroxy, or C₁₋₅ alkyl.

The above compounds may be administered alone or in combination with other feed additive agents. The above vitamin D compounds or combinations thereof can be readily administered in amounts of from 0.1 μg/kg to 100 μg/kg of feed either by mixing them directly into animal feed or by mixing them into a feed supplement or additive which in turn may be mixed directly into the animal feed or fed to the animal separately from the feed. Also, the compounds may be administered by separate oral dosage, by injection or by transdermal means or in combination with other 1α-hydroxylated vitamin D compounds, the proportions of each of the compounds in the combination being dependent upon the particular problem being addressed and the degree of response desired, are generally effective to practice the present invention. In dairy cows, the preferred dosage is 75 μg per day of 1α-hydroxyvitamin D₃. Amounts in excess of about 100 micrograms per day or the combination of that compound with other 1α-hydroxylated vitamin D compounds, are generally unnecessary to achieve the desired results, may result in hypercalcemia, and may not be an economically sound practice. It should be understood that the specific dosage administered to any given animal will be adjusted in accordance with the specific compounds being administered, the problem to be treated, the condition of the animal and the other relevant facts that may modify the activity of the compound or the response of the animal, as is well known by those skilled in the art. In general, either a single dose or divided daily dosages may be employed, as is well known in the art.

If administered separately from the animal feed, dosage forms of the various compounds can be prepared by combining them with non-toxic pharmaceutically acceptable carriers to make either immediate release or slow release formulations, as is well known in the art. Such carriers may be either solid or liquid such as, for example, corn starch, lactose, sucrose, peanut oil, olive oil, sesame oil and propylene glycol. If a solid carrier is used, the dosage form of the compounds may be tablets, capsules, powders, troches or lozenges or top dressing as micro-dispersible forms. If a liquid carrier is used, soft gelatin capsules, or syrup or liquid suspensions, emulsions or solutions may be the dosage form. The dosage forms may also contain adjuvants, such as preserving, stabilizing, wetting or emulsifying agents, solution promoters, etc. They may also contain other therapeutically valuable substances.

The present invention also relates to an animal feed composition for a dairy cow and method of compounding an animal feed utilizing a 1α-hydroxylated vitamin D compound to lower the dietary requirement of phosphorus in the cow's feed. The 1α-hydroxylated vitamin D compounds suitable for this use have been previously described herein. The amount of an inorganic phosphorus supplement (18.5%P) that is typically incorporated with the feed may be reduced to 0.3% or less by weight or may be entirely eliminated from the cow's diet. This beneficial reduction in phosphorus is a direct result of the incorporation of a 1α-hydroxylated vitamin D compound in the animal feed.

The animal feed may be any protein-containing organic meal normally employed to meet the dietary requirements of a dairy cow. Many of such protein-containing meals are typically primarily composed of corn, soybean meal or a corn/soybean meal mix. For example, a typical commercially available diet fed to dairy cows is set forth in Table 1. The diet in Table 1 is a typical example of an animal feed with which the present 1α-hydroxylated vitamin D compounds may be incorporated to reduce the amount of phosphorus excrement in manure. Thus, any type of protein-containing organic meal typically fed to a dairy cow may be utilized as the base mix to which the 1α-hydroxylated vitamin D compounds of the present invention may be incorporated.

The present invention is applicable to the diet of numerous ruminant animals, which herein is defined as including multigastric mammals having a complex 2- or 4-chambered stomach. In particular, the diet may be employed with commercially significant milk-producing ruminants such as dairy cows.

In a method of compounding feed for a dairy cow in accordance with the present invention, the 1α-hydroxylated vitamin D compounds utilized is incorporated with the animal feed in an amount so as to provide to the animal from about 5 μg to about 100 μg per day of the compound. The preferred amount is 75 μg per day for diary cows, and the preferred compound is 1α-hydroxyvitamin D₃. The feed mixture is then fed as a mash or is formed into desired discrete shapes for further processing and packaging. In general, these discrete shapes may be pellets, blocks or briquettes formed by known extrusion and/or compacting techniques. The particular processing technique utilized does not affect the performance of the 1α-hydroxyated vitamin D compounds in the animal feed mixture.

The present invention is more specifically described by the following examples, which are meant to be illustrative only.

### Dairy Cow Efficacy Trial

A. Objective

To demonstrate that feeding 75 μg of 1-α-OH vitamin D₃ daily to lactating cows increases phosphorus (P) uptake from the gut, as evidenced by a consequent increase in blood serum P and a decrease in fecal P excretion.

B. Procedures

Eight multiparous lactating dairy cows (about 150 days in milk at start of the experiment) were blocked into two groups according to milk yield. Cows in each block were assigned randomly to four different treatments. The experimental design was a 4×4 Latin Square. Each period was four weeks in length. The first three weeks were used as an adaptation period, and measurements were taken during the last week of each period.

The four treatments were:

1) High P diet. Dietary P at 0.47% (DM basis)

2) Diet 1 plus 1-α-OHD₃ (75 μg/cow/day)

3) Low P diet. Dietary P at 0.35% (DM basis)

4) Diet 3 plus 1-α-OHD₃ (75 μg/cow/day).

The diets (Table 1) were fed ad libitum as a total mixed ration once daily. The cows were housed in individual tie stalls. Daily feed offered and refused was recorded for individual cows. Feed refusals were restricted to 10% of intake on an as-fed basis. Daily samples of silage and refusals were composited weekly for chemical analysis. Samples of individual feed ingredients were collected once weekly. The dry matter content of feed ingredients was determined by oven drying at 60° C. for 48 hr. Diet formulations were adjusted weekly, if necessary, to account for changes in dry matter content of diet ingredients. All of the feed ingredients were analyzed for chemical composition. Alfalfa and corn silage were analyzed weekly for neutral detergent fiber (NDF), acid detergent fiber (ADF), and crude protein (CP). High moisture ear corn (HMEC), soybean meal (SBM), roasted soybean meal (RSB), and blood meal (BM) were composited every four weeks (one sample per period) and analyzed for CP.

Cows were weighed two days in a row before the trial started and two days in a row when it finished. Daily milk weights were recorded. All cows were injected with bovine somatotropin (Posilac®). Blood samples were collected on the last day of weeks 1, 2, and 3 of each period. During week 4, blood samples were obtained on each of the last three days. Blood was obtained 5 hours after feeding from the coccygeal vein or artery in a gel and clot co-activator vacutainer. Samples were centrifuged the next day to separate the serum. Inorganic phosphorus and calcium analyses were performed on serum samples by Marshfield Laboratories, Marshfield, Wis.

During the last two days of each period, six urine samples were collected at 8 am, 4 pm, and 11 pm on the first day and 12 noon, 8 pm, and 6 am on the second day. Urine samples were then composited for phosphorus, calcium, and creatinine analysis. Creatinine was measured to enable an estimate of urine output. Milk samples were obtained during four days of each period. The milk sample was split; one-half was mixed with a preservative, and the other half was refrigerated without a preservative. The samples with preservative were analyzed for milk composition by Wisconsin Ag Source Milk Analysis Laboratory (Menomonie, Wis.), and the samples without preservative were composited according to milk yield and analyzed for mineral composition in the Soil and Plant Analysis Laboratory, Department of Soil Science, University of Wisconsin-Madison.

All feed ingredients were sampled several times during each period, and one composite sample for each ingredient was generated per period. The composite samples were analyzed for mineral composition at the Soil and Plant Analysis Laboratory cited above.

Feed DM digestibility was determined using Yb as an external marker. A Yb solution, prepared by dissolving 2.24 g of YbCl₃ (1 g of Yb) in 30 ml of water was sprayed onto 500 g of alfalfa silage (DM basis). The Yb-marked alfalfa silage was mixed into the total mixed ration to give 40 mg of Yb/kg of dietary DM. The total mixed ration labeled with Yb was fed for nine days. During the last two days of Yb feeding (last two days of each period), eight fecal grab samples were collected from each cow at 7 am, 12 noon, 6 pm, and 11 pm on the first day and at 9 am, 3 pm, 9 pm, and 5 am on the second day. Fecal samples were dried at 60° C. for 72 h and ground through a 2-mm Wiley mill screen. Fecal samples were dry-ashed in duplicate (Combs and Satter, 1992). During the last four days of each period, feed refusals for each cow were collected and composited into one sample per cow per period. These samples were dry-ashed in the same way as feces, and analyzed for Yb. Concentrations of Yb (ppm) in feed, feed refusals, and fecal samples were determined by direct current plasma spectroscopy [(Combs and Satter, 1992); Spectra Metrics, Inc., subsidiary of Beckman Instruments, Inc., Andover, Mass.]. The DM digestibility (percentage) for individual cows was calculated as follows: DM digestibility (%)=(1-concentration of Yb in DM consumed/average concentration of Yb in each cow's fecal samples)100. Digestibility of dietary P in different treatments was calculated using P input-output data.

C. Results

The effects of 1-α-OH vitamin D₃ on P utilization by dairy cows is shown in Table 2. Feed consumption (DM intake) was not affected by either dietary P concentration or the presence of 1-α-OH vitamin D₃. Fecal P excretion was reduced by daily inclusion of 75 μg of 1-α-OH vitamin D₃ in the cows' diets.

Phosphorus excretion in manure was reduced by about 10 g per day for both the high- and low-P treatments. This equates to a 14% reduction in fecal P excretion due to supplementation of 1-α-OH vitamin D₃. Milk production was unaffected by supplementation of 75 μg of 1-α-OH vitamin D₃.

When fed conventional diets, dairy cows would normally and typically be expected to produce from about 35 to 45 kg milk/day. The data in Table 2 confirms this as one can see that dairy cows fed a diet containing 0.47% P, with or without 1α-OH-D₃ supplementation, produced normal yields of 36.9 kg milk/day and 37.8 kg milk/day respectively. Dairy cows fed a diet containing only 0.35% P, with or without 1α-OH-D₃ supplementation, maintained milk production at these normal yields, i.e. produced 37.6 kg milk/day and 37.3 kg milk/day respectively.

Milk composition is shown in Table 3. Milk composition was similar for all treatments, but there were three small changes in response to the 1-α-OH vitamin D₃ treatment that were statistically significant or approached significance. Milk lactose was increased slightly with the 1-α-OH vitamin D₃ treatment, and potassium was decreased with the same treatment. Milk protein may have been decreased slightly with the 1-α-OH vitamin D₃ treatment, but the change was very small.

The effects of 1-α-OH vitamin D₃ on concentrations of P and calcium (Ca) in blood serum and urine are shown in Table 4. The effect of 1-α-OH vitamin D₃ was to increase the concentration of both P and Ca in blood serum. This was a highly significant effect and occurred with both the high- and low-P treatments. The excretion in urine of both P and Ca was low. There is a tendency for some dairy cattle to excrete slightly more P in the urine when P is fed in excess of requirement, and that was evident in this experiment when the high-P treatment resulted in an increase in urinary P. However, this represented only about 2% of dietary P intake, even at a concentration of 5.15 mg P per dl of urine. Calcium excretion via urine was increased with supplementation of 1-α-OH vitamin D₃.

D. Summary

Supplementation of 1-α-OH vitamin D₃ increased P uptake from the gut as evidence by decreased excretion of P in the feces and by increased P concentration in blood serum.

TABLE 1
Ingredient Composition of Diets

| | High P | Low P |
|---|---|---|
| | (% DM basis) | (% DM basis) |
| Ingredient | | |
| Alfalfa silage | 25.0 | 25.0 |
| Corn silage | 25.0 | 25.0 |
| High moisture ear corn | 31.85 | 32.25 |
| Soybean meal | 7.7 | 7.7 |
| Roasted soybeans | 6.5 | 6.5 |
| Blood meal | 2.0 | 2.0 |
| Sodium monophosphate | 0.4 | — |
| Calcium carbonate | 1.0 | 1.0 |
| Magnesium oxide | 0.05 | 0.05 |
| Trace-mineralized salt¹ | 0.5 | 0.5 |
| Vitamin supplement | Trace | Trace |
| Chemical Composition | | |
| NE_{L}³, Meal/kg DM | | |
| Crude protein, % DM | 17.85 | 17.88 |
| Undegraded protein, % DM | 6.87³ | 6.87³ |
| NDF⁴ (%) | 19.55 | 19.55 |
| ADF⁴ (%) | 14.70 | 14.70 |

¹Contained salt (94.0% minimum or 96.0% maximum); zinc (5.5 ppm); manganese (5.5 ppm); copper (1.4 ppm); iron (3.45 ppm); iodine (80 ppm); cobalt (20 ppm); and selenium (60 ppm).
²To provide 150,000 IU vitamin A, 50,000 IU vitamin D, and 500 IU vitamin E per cow per day.
³Estimated from NRC tables.
⁴NDF and ADF are from forages only (alfalfa silage and corn silage).

TABLE 2
Effects of 1-α-OHD₃ on Phosphorus Utilization by Lactating Dairy
Cows

| | | | | | P Value | |
|---|---|---|---|---|---|---|
| | Treatments | | | | High vs. | 1-α-OH |
| Items | 1 | 2 | 3 | 4 | Low P | Vitamin D₃ |
| Diet, % P | 0.47 | 0.47 | 0.35 | 0.35 | — | — |
| 1-α-OHD₃, | 0.0 | 75.0 | 0.0 | 75.0 | — | — |
| μg/day | | | | | | |
| Dry matter | 24.6 | 23.8 | 23.8 | 24.1 | 0.53 | 0.44 |
| intake, kg/day | | | | | | |
| P intake, g/day | 115.6 | 111.9 | 83.3 | 84.4 | — | — |
| Fecal DM | | | | | | |
| Kg/day | 7.2 | 7.2 | 7.2 | 7.2 | — | — |
| P, % | 1.15 | 0.99 | 0.90 | 0.77 | 0.13 | 0.09 |
| P, g/day | 82.8 | 71.3 | 64.8 | 55.4 | — | — |
| Milk | | | | | | |
| Kg/day | 37.8 | 36.9 | 37.3 | 37.6 | 0.90 | 0.63 |
| P, ppm | 1030 | 1034 | 1050 | 1021 | 0.79 | 0.36 |
| P, g/day | 38.9 | 38.2 | 39.2 | 38.4 | — | — |
| Milk and Fecal P | | | | | | |
| g/day | 121.7 | 109.5 | 104.0 | 93.8 | — | — |

TABLE 3
Effect of 1-α-OH D₃ on Milk Composition

| | | | | | P Value | |
|---|---|---|---|---|---|---|
| | Treatment | | | | High vs. | 1-α-OH |
| Item | 1 | 2 | 3 | 4 | Low P | Vitamin D₃ |
| Milkfat, | 3.41 | 3.45 | 3.30 | 3.54 | 0.89 | 0.22 |
| % | | | | | | |
| Milk | 3.33 | 3.28 | 3.36 | 3.31 | 0.41 | 0.08 |
| protein, | | | | | | |
| % | | | | | | |
| Lactose, | 4.91 | 4.98 | 4.86 | 4.93 | 0.08 | 0.02 |
| % | | | | | | |
| Phos- | 1030 | 1034 | 1050 | 1021 | 0.79 | 0.36 |
| phorus, | | | | | | |
| ppm | | | | | | |
| Potas- | 1639 | 1595 | 1666 | 1559 | 0.83 | 0.002 |
| sium, | | | | | | |
| ppm | | | | | | |
| Calci- | 1205 | 1238 | 1230 | 1248 | 0.49 | 0.34 |
| um, | | | | | | |
| ppm | | | | | | |
| Magne- | 108 | 108 | 109 | 105 | 0.45 | 0.17 |
| sium, | | | | | | |
| ppm | | | | | | |

TABLE 4
Effect of 1-α-OH Vitamin D₃ on Phosphorus and Calcium Concentrations
in Blood Serum and Urine

| | | | | | P Value | |
|---|---|---|---|---|---|---|
| | Treatment | | | | High vs. | 1-α-OH |
| Item | 1 | 2 | 3 | 4 | Low P | Vitamin D₃ |
| Blood serum | | | | | | |
| Phosphorus | 5.56 | 8.77 | 5.23 | 7.63 | 0.03 | 0.0001 |
| (mg/dl) | | | | | | |
| Calcium | 8.94 | 10.32 | 8.79 | 10.14 | 0.34 | 0.0001 |
| (mg/dl) | | | | | | |
| Urine | | | | | | |
| Phosphorus | 1.23 | 5.15 | 0.79 | 0.40 | 0.08 | 0.23 |
| (mg/dl) | | | | | | |
| Calcium | 3.10 | 12.03 | 3.56 | 15.46 | 0.14 | 0.0001 |
| (mg/dl) | | | | | | |

We claim:

1. A method of maintaining milk production in a dairy cow fed a low phosphorus diet, comprising the steps of:
feeding a feed that contains about 0.3% by weight or less of an inorganic phosphorus supplement to a dairy cow; and
feeding with said feed an effective amount of a 1α-hydroxylated vitamin D compound for increasing phosphorus uptake in the cow's gut.

2. The method of claim 1 wherein said 1α-hydroxylated vitamin D compound is fed as a top dressing on said feed.

3. The method of claim 1 wherein said effective amount of the 1α-hydroxylated vitamin D compound comprises about 0.1 μg/kg to about 100 μg/kg of diet.

4. The method of claim 1 wherein the feed contains 0% by weight of an inorganic phosphorus supplement.

5. The method of claim 1 wherein said 1α-hydroxylated vitamin D compound is characterized by the following general structure: 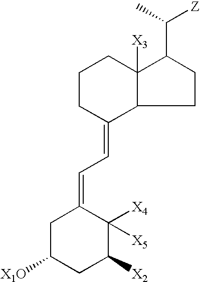
where X₁ may be hydrogen or a hydroxy-protecting group, X₂ may be hydroxy, or protected hydroxy, X₃ may be hydrogen or methyl, X₄ and X₅ each represent hydrogen or taken together X₄ and X₅ represent a methylene group, and where Z is selected from Y, —OY, —CH₂OY, —C≡CY and —CH═CHY, where the double bond may have the cis or trans stereochemical configuration, and where Y is selected from hydrogen, methyl, —CR₅O and a radical of the structure: 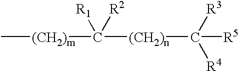
where m and n, independently, represent integers from 0 to 5, where R¹ is selected from hydrogen, hydroxy, protected-hydroxy, fluoro, trifluoromethyl, and C₁₋₅-alkyl, which may be straight chain or branched and, optionally, bear a hydroxy or protected-hydroxy substituent, and where each of R², R³ and R⁴, independently, is selected from hydrogen, fluoro, trifluoromethyl and C₁₋₅ alkyl, which may be straight-chain or branched, and optionally bear a hydroxy or protected-hydroxy substituent, and where R¹ and R², taken together, represent an oxo group, or an alkylidene group, ═CR₂R₃, or the group —(CH₂)ₚ—, where p is an integer from 2 to 5, and where R³ and R⁴, taken together, represent an oxo group, or the group —(CH2)_{q}—, where q is an integer from 2 to 5, and where R⁵ represents hydrogen, hydroxy, protected-hydroxy, or C₁₋₅ alkyl.

6. The method of claim 1 wherein the vitamin D compound is 1α-hydroxyvitamin D₃.

7. The method of claim 1 wherein the vitamin D compound is 1α,25-dihydroxyvitamin D₃.
